(19) United States
(12) Patent Application Publication
(10) Pub. No.: US 2025/0090587 A1
(43) Pub. Date: Mar. 20, 2025

(54) PLATELET-DERIVED MITOCHONDRIA-CONTAINING EXTRACELLULAR VESICLES FOR USE IN THE TREATMENT OF OCULAR DISORDERS

(71) Applicant: MITRIX BIO, INC, Pleasanton, CA (US)

(72) Inventor: Thomas B. Benson, Los Altos, CA (US)

(21) Appl. No.: 18/789,363

(22) Filed: Jul. 30, 2024

Related U.S. Application Data
(63) Continuation of application No. PCT/US2023/011888, filed on Jan. 30, 2023.
(60) Provisional application No. 63/267,422, filed on Feb. 1, 2022.

(51) Int. Cl. A61K 35/19; C12N 5/078

(52) U.S. Cl. CPC A61K 35/19; C12N 5/0644

(57) ABSTRACT

Platelet-derived extracellular vesicles that include mitochondria (PEVs) are used in the treatment of an ocular disorder, or a symptom of the ocular disorder. The PEVs are collected by obtaining blood from one or more donors, adding an anticoagulant and a buffer to the blood to form a mix, separating the mix into supernatant and platelet rich plasma (PRP), collecting the PRP, stimulating the collected PRP, thereby expelling extracellular vesicles from platelets in the PRP, and collecting the extracellular vesicles as PEVs.

### RELATED APPLICATION INFORMATION

This application is a continuation of PCT Application Serial No. PCT/US2023/011888, filed Jan. 30, 2023, which claims priority to U.S. Provisional Application Ser. No. 63/267,422, filed Feb. 1, 2022. Each of the aforementioned applications are incorporated herein by reference in their entireties.

### BACKGROUND OF THE INVENTION

### Field

The presently disclosed and claimed inventions relate generally to use of platelet-derived mitochondria-containing extracellular vesicles (PEVs) for treating ocular disorders via delivery into the intraocular space of an eye.

### Description

Mitochondria are membrane-limited subcellular organelles that contain their own DNA (mtDNA) and their own machinery for synthesizing RNA and proteins. They are found in nearly all eukaryotic cells and vary in number and location depending on the cell type.

Mitochondria perform numerous essential tasks in the eukaryotic cell such as pyruvate oxidation, the Krebs cycle and metabolism of amino acids, fatty acids, and steroids. The primary function of mitochondria is the generation of energy as adenosine triphosphate (ATP) by means of the electron-transport chain and the oxidative-phosphorylation system (the “respiratory chain”). Additional processes in which mitochondria are involved include heat production, storage of calcium ions, calcium signaling, programmed cell death (apoptosis) and cellular proliferation. It has been disclosed that mitochondria have a role in cell regulatory and signaling events (i.e. regulation of Ca²⁺ fluxes, oxidative stress and energy-related signaling among others).

Therefore, mitochondrial dysfunction is an underlying factor in multiple diseases including cardiovascular disease, cancer. Alzheimer's, diabetes, vision loss, and frailty. Vision loss can be the result from a number of ocular disorders such as aged macular degeneration (AMD), retinitis pigmentosa (RP). Leber's Hereditary Optic Neuropathy (LHON), glaucoma, and diabetic retinopathy. While this list is not exhaustive, one aspect shared between these disorders mitochondrial dysfunction giving rise to pathology of vision loss or loss of vision acuity. For example, retinal pigment epithelium (RPE) cells are thought to be a key factor in AMD pathology. It is believed that excessive production of reactive oxygen species (ROS) due to mitochondrial dysfunction in RPE cells can lead to damage and, ultimately, to the degeneration of RPE cells in the retina. Mitochondrial dysfunction in AMD is thought to be brought upon by accumulated damage from ROS to the mitochondrial DNA (mtDNA) over time. Because one of the main functions of RPE cells is to maintain the health of photoreceptors, the degeneration of the former leads to the dysfunction of latter, which results in visual perturbations that are associated with AMD. Another example is LHON, which is known to be a hereditary mitochondrial genetic disorder that is manifested by three primary mitochondrial DNA (mtDNA) mutations in 90% of the cases. From these mtDNA mutations. LHON primarily affects retinal ganglion cells (RGC) causing their degeneration, which leads to vision loss. The degeneration and eventual loss of RGCs from the inner retina are cellular hallmarks of this disorder.

Some have approached the problem of treating these ocular disorders by using gene therapy in the hopes of correcting the damaged or mutated mtDNA. However, genotoxic adverse reactions from such an approach can present safety-related issues. An alternative approach is to transfuse healthy mitochondria into cells affected by such ocular disorders. The technique of mitochondrial transfusion—gathering mitochondria from an outside source and transfusing into the body—has recently been developed by a number of major universities. While mitochondrial transfusion is generally less invasive than other approaches, finding a source of mitochondria and preparing the mitochondria for transfusion has long been a challenge.

### SUMMARY

The methods disclosed herein each have several aspects, no single one of which is solely responsible for their desirable attributes. Without limiting the scope of the claims, some prominent features will now be discussed briefly. Numerous other embodiments are also contemplated, including embodiments that have fewer, additional, and/or different components, steps, features, objects, benefits, and advantages. The components, aspects, and steps may also be arranged and ordered differently. After considering this discussion, and particularly after reading the section entitled “Detailed Description”, one will understand how the features of the devices and methods disclosed herein provide advantages over other known devices and methods.

In some embodiments, a method of treatment for an ocular disorder, or a symptom of the ocular disorder, in a patient in need thereof is provided, the method including obtaining platelet-derived extracellular vesicles that include mitochondria (PEVs), wherein the PEVs have been collected by: obtaining blood from one or more donors: adding an anticoagulant and a buffer to the blood to form a mix: separating the mix into supernatant and platelet rich plasma (PRP); collecting the PRP; stimulating the collected PRP, thereby expelling extracellular vesicles from platelets in the PRP; and collecting the extracellular vesicles as the PEVs. In some embodiments, the method further includes administering an effective amount of the PEVs to an eye of the patient, thereby treating the ocular disorder. In some embodiments, the PEVs have been collected at a different site than a site where the treatment is carried out.

In some embodiments, the administering includes delivering the PEVs from a syringe that is configured to deliver the PEVs into the eye. In some embodiments, the administering includes delivering the PEVS via a port delivery system that provides a PEV implant for prolonged delivery of the PEVs into the eye. In some embodiments, the administering includes delivering the effective amount of the PEVs into a vitreous chamber of the eye.

In some embodiments, the collected PRP is stimulated with immune complexes in presence of Ca2+. In some embodiments, the immune complexes include heat-aggregated IgG. In some embodiments, the collected PRP is stimulated by freeze-thaw cycles. In some embodiments, a concentration of the heat-aggregated IgG is about 0.1 mg/mL to about 2.5 mg/mL, and wherein concentration of the Ca2+ is about 1 mM to about 25 mM. In some embodiments, the anticoagulant is anticoagulant citrate dextrose (ACD). In some embodiments, the buffer is Tyrode's buffer at about pH 6 to about pH 7. In some embodiments, the separating step is conducted by centrifuge. In some embodiments, the blood has been stored for four or more days. In some embodiments, the blood has been stored for up to one year.

In some embodiments, during and/or after the administering the PEVs into the eye, the PEVs contact at least one cell of the eye. In some embodiments, the PEVs are internalized into the cell after the PEVs contact the cell. In some embodiments, the effective amount corresponds to an amount of the internalized PEVs, which ranges from about 3 PEV/cell to about 100 PEV/cell. In some embodiments, the PEVs are frozen while stored. In some embodiments, the frozen PEVs are stored in combination with a cryoprotectant. In some embodiments, the cryoprotectant is selected from the group consisting of a saccharide, an oligosaccharide, and a polysaccharide.

In some embodiments, the ocular disorder is aged macular degeneration. In some embodiments, the ocular disorder is retinitis pigmentosa. In some embodiments, the ocular disorder is Leber's Hereditary Optical Neuropathy. In some embodiments, the ocular disorder is diabetic retinopathy. In some embodiments, the ocular disorder is glaucoma. In some embodiments, the cell includes a retinal pigment epithelium cell. In some embodiments, the cell includes a retinal ganglion cell. In some embodiments, the cell is located about a macula of the eye

### BRIEF DESCRIPTION OF THE DRAWINGS

Features of examples of the present disclosure will become apparent by reference to the following detailed description and drawings, in which like reference numerals correspond to similar, though perhaps not identical, components. For the sake of brevity, reference numerals or features having a previously described function may or may not be described in connection with other drawings in which they appear.

FIG. 1 is a schematic diagram showing a method of obtaining PEVs according to some embodiments.

FIGS. 2A and 2B are fluorescent images of retinal pigmented epithelium cells (RPEC) grown in culture showing uptake of PEVs into RPECs; FIG. 2A shown at 20× and FIG. 2B shown at 40×.

FIG. 3 are fluorescent images that show uptake and internalization of PEVs into cultured RPECs.

FIG. 4 shows fluorescent images demonstrating the uptake of DsRed-labeled PEVs/mitlets by RPECs in vitro after 3 and 6 hours.

FIG. 5 is a fluorescent image showing internalized PEV-delivered mitochondria in cultured RPECs.

FIGS. 6A and 6B illustrate the uptake of PEVs of various effective amounts into different cell types, wherein FIG. 6A illustrates the uptake of PEVs into RPECs and FIG. 6B illustrates the uptake of PEVs into brain endothelial cells (bEND).

FIGS. 7A-7D show the oxygen consumption rate (OCR) at various stages of oxidative phosphorylation in the mitochondria of the RPECs where at least some of the mitochondria were transfused into the RPECs from PEVs at various effective amounts.

FIG. 8 shows the OCR of RPECs, with respect to basal respiration, that receive various amounts of naked mitochondria.

FIG. 9 are fluorescent images showing internalization of PEVs into cells of the bone marrow and spleen in vivo.

FIG. 10 is a schematic diagram showing a method of treatment for an ocular disorder according to some embodiments.

FIG. 11 is a dot plot that represents PEV populations where the PEVs, labeled with DsRed, are represented as approximately 40% of the total CD41+PEVs.

### DETAILED DESCRIPTION

In the Summary Section above and the Detailed Description Section, and the claims below, reference is made to particular features of the invention. It is to be understood that the disclosure of the invention in this specification includes all possible combinations of such particular features. For example, where a particular feature is disclosed in the context of a particular aspect or embodiment of the invention, or a particular claim, that feature can also be used, to the extent possible, in combination with and/or in the context of other particular aspects and embodiments of the invention, and in the invention generally.

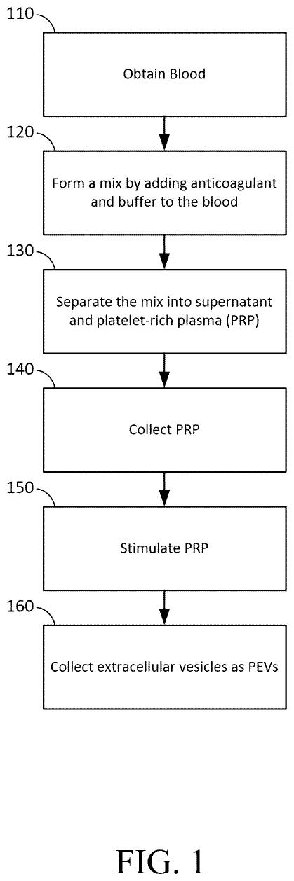

As shown in FIG. 1, some embodiments relate to a method of extracting platelet-derived mitochondria-containing extracellular vesicles (PEVs). In some embodiments, the methods include 1) obtaining blood from donors in step 110, 2) adding anticoagulant and a buffer to the blood to form a mix in step 120, 3) separating the mix into supernatant and platelet rich plasma (PRP) in step 130, 4) collecting platelet rich plasma (PRP) in step 140, 5) stimulating the collected platelets in step 150, and 6) collecting the PEVs in step 160.

Finding a source of mitochondria to transplant is a challenge. Just like any donated organ, mitochondria from young healthy donors are in short supply. Some diseases or injuries might be cured by autologous mitochondria—removed from a leg muscle in one's own body for example—however, for many other diseases, the “patients” have poor quality mitochondria due to age or mutation to mitochondrial DNA (mtDNA). For these patients, donated mitochondria are a preferred solution. In addition, freshly fully isolated mitochondria die quickly within minutes of isolation and may also provoke immune reactions when put naked into the bloodstream, reducing their effectiveness as a therapy. Therefore, it would be convenient to find an easy and readily available source of donation-ready mitochondria, which at the same time, are encased in some sort of coating, vesicle, or vehicle suspension (or any combination thereof) that protect them from the immune system.

A platelet from human blood contains 4-5 mitochondria on average that are expelled in extracellular vesicles when platelets are activated. The platelet-derived mitochondria-containing extracellular vesicles are referred to PEVs herein. These PEVs are usually larger (>400 nM), and less well-known than other platelet extracts or lysates (30-100 nM), however other sizes may also apply.

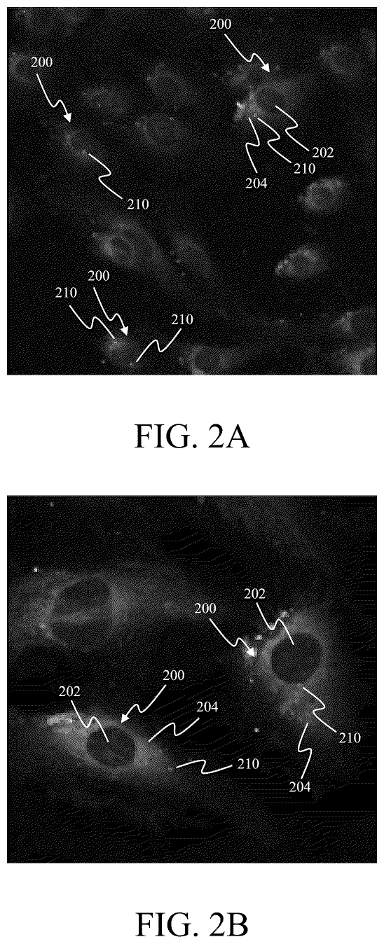

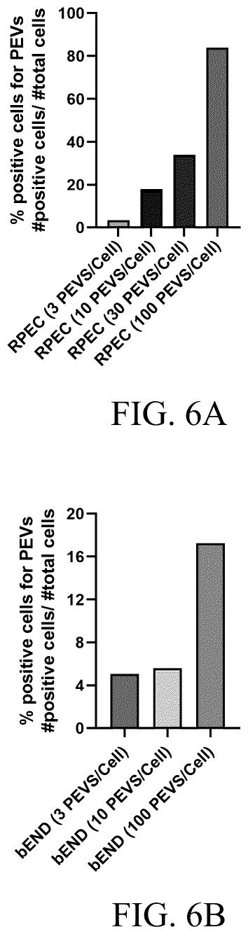

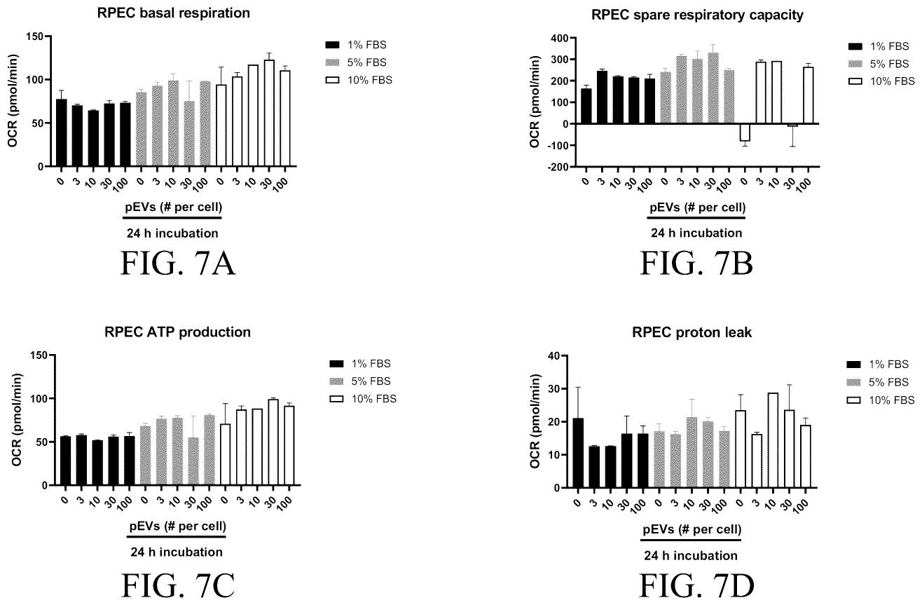

PEVs have been shown to donate their mitochondria to cells nearby (shown in FIGS. 2A-2B. 3, 4, and 5) with different cells uptaking the PEVs (shown in FIGS. 6A and 6B) that can increase the respiratory activity of the cells that absorb them as shown in FIGS. 7A-7D, thus regenerating tissue and curing several diseases of aging. PEVs have several advantages for fast commercialization: notably, they can be extracted from donated platelets that have “expired” and must be thrown away; they represent another good medically-valid use for platelets which otherwise might go to waste; they could be collected at most blood banks, who already have all the needed skilled personnel, clean handling practices, and equipment needed, and are already in close proximity to hospitals, thus making PEV product potentially available to world-wide use extremely soon. PEVs are a variety of platelet transfusion and therefore are more likely to be embraced and tested by medical professionals who are already familiar with blood transfusion therapies. Furthermore. PEVs can be prepared for localized transfusion into various internal anatomical regions to treat various clinical disorders using delivery devices already on the market.

In some embodiments, a non-limiting example of a delivery device is a syringe that includes at least a hollow barrel that forms an internal space, a plunger that is coupled and fitted into the hollow barrel, and a needle that is coupled to the barrel, the needle including a space that is contiguous with the internal space of the hollow barrel when the needle is coupled with the barrel. Both the plunger and the needle may be either directly or indirectly coupled to the hollow barrel. The syringe is constructed to deliver the PEVs and/or naked mitochondria intraocularly or intravitreally. The syringe may also be constructed to deliver the PEVs and/or naked mitochondria subretinally. In some embodiments, the syringe is constructed to deliver the PEVs and/or naked mitochondria subcutaneously. In some embodiments, syringe is constructed to deliver the PEVs and/or naked mitochondria into the peritoneal cavity (intraperitoneal injection). In some embodiments, the syringe is constructed to deliver the PEVs and/or naked mitochondria systemically to the patient.

In some embodiments, a non-limiting example of a delivery device is a port delivery system, which provides sustained release of PEVs and/or naked mitochondria (any of which may be optionally combined with other therapeutic agents) via intraocular or intravitreal delivery thereof. The port delivery system has been described by U.S. Pat. No. 9,968,603, the disclosure of which is hereby incorporated by reference.

As but one non-limiting example in some embodiments. Gyroscope Therapeutics, has developed the ORBIT™ subretinal delivery system that can be adapted for delivery of PEVs. Another delivery system involves providing the PEVs on contact-lenses, which are then placed in the affected eye. The PEVs can also be embedded in a gel-like material and deployed in “microneedles” as described by Lee et al., Advanced Functional Materials, doi.org/10.1002/adfm.202000086 (2020), the disclosure of which is hereby incorporated by reference. Many other such delivery systems are known and can be adapted to deliver PEVs.

In some embodiments, the blood is derived from a mammalian subject. According to another embodiment, the mammalian subject is a human subject. According to another embodiment, the mammalian subject is selected from a group consisting of: a human, a horse, a dog, a cat, a mouse, a rat, a cow and a sheep. Each possibility represents a separate embodiment of the present invention. According to another embodiment, the PEVs of the invention are derived from a mammalian cell. According to another embodiment, the mammalian cell is a human cell. According to another embodiment, the PEVs are derived from cells in culture. According to another embodiment, the PEVs are derived from a tissue.

According to another embodiment, the PEVs are derived from a cell or a tissue selected from the group consisting of: human placenta, human placental cells grown in culture and human blood cells. According to another embodiment, the PEVs of the invention are derived from a cell or a tissue selected from the group consisting of: placenta, placental cells grown in culture, and blood cells. According to another embodiment, naked mitochondria may be isolated from a cell or a tissue selected from the group consisting of: liver, bone marrow, placenta, human placental cells, or any other tissues of a donor. According to another embodiment, naked mitochondria may be isolated from a cell grown in culture or a tissue grown in culture selected from the group consisting of: liver, bone marrow, placenta, human placental cells, or any other tissues of a donor.

As used herein, the phrase “naked mitochondria” refers to mitochondria that are isolated from the cell or the tissue. In some embodiments the cell is a cell grown in culture. In some embodiments, the tissue is tissue grown in culture. The naked mitochondria can be suspended in a freezing buffer, a hydrogel, a pharmaceutically acceptable liquid medium capable of supporting of the naked mitochondria, or a buffer solution which includes a saccharide. In some embodiments, the hydrogel is biocompatible, biodegradable, and capable of supporting naked mitochondria. In some embodiments, the hydrogel may be thermosensitive, which includes temperature-dependent hydrophilicity and hydrophobicity. In some embodiments, the hydrogel is biocompatible, biodegradable, capable of supporting naked mitochondria, and thermosensitive, the latter of which includes the hydrogel having temperature-dependent hydrophilicity and hydrophobicity.

As used herein, the phrases “cells grown in culture” or “a tissue grown in culture” refers to a multitude of cells or a tissue, respectively, grown in a liquid, semi-solid or solid medium, outside of the organism from which the cells or tissue derive. According to some embodiments, cells grown in culture are cells grown in bioreactors. According to a non-limiting example, cells may be grown in a bioreactor, followed by isolation of PEVs from the cells. According to another non-limiting example, cells may be grown in a bioreactor, which is followed by isolation of the mitochondria from the cells. In some embodiments, the isolated mitochondria from the cells grown in a bioreactor is naked mitochondria.

In some embodiments, the blood is from mice. Mouse blood is used to test the feasibility of the method of extracting PEVs. In some embodiments, the blood is from human donors.

Once blood is obtained in step 110, anticoagulant and a buffer are added in step 120 to prevent blood from becoming thick and solid. In some embodiments, the anticoagulant is ACD (20%). In some embodiments, the buffer is 40% Tyrode's buffer having a pH of about 6 to about 7, preferably pH 6.5.

After adding anticoagulant and a buffer to blood in step 120, the mixture is then separated into supernatant and platelet rich plasma (PRP) in step 130. In some embodiments, the separating is by centrifuging. Plasma is the liquid portion of whole blood. It is composed largely of water and proteins, and it provides a medium for red blood cells, white blood cells and platelets to circulate through the body. Platelets are blood cells that cause blood clots and other necessary growth healing functions. After the centrifuging of the mixture, blood cells are formed a pellet that accumulates at the bottom of a tube. The pellet is referred to as platelet rich plasma (PRP), which contains concentrated platelets. The pellet is then collected in step 140.

Buffers are then added to the collected PRP from step 140 to resuspend the platelets. The platelets are then activated or stimulated in step 150. There are many ways to activate platelets. Any of a number of substances can be used for this purpose including carbon radioisotopes, prostaglandins, serotonin, adenosine triphosphate, collagen. 1-lactate dehydrogenase, thrombin, magnesium, adenosine, calcium and heat-aggregated antibodies. In some embodiments, platelets are activated by freeze-thaw cycles. As used herein, the term “freeze-thaw cycle” refers to freezing of the mitochondria of the invention to a temperature below 0° C.) maintaining the mitochondria in a temperature below 0° C. for a defined period of time and thawing the mitochondria to room temperature or body temperature or any temperature above 0° C. The term “room temperature”, as used herein refers to a temperature of between 18° C., and 25° C. The term “body temperature”, as used herein, refers to a temperature of between 35.5° C., and 37.5° C. preferably 37° C.

In another embodiment, mitochondria that have undergone a freeze-thaw cycle were frozen at a temperature of at least −70° C. In another embodiment, the mitochondria that have undergone a freeze-thaw cycle were frozen at a temperature of at least −20° C. In another embodiment, the mitochondria that have undergone a freeze-thaw cycle were frozen at a temperature of at least −4° C. In another embodiment, the mitochondria that have undergone a freeze-thaw cycle were frozen at a temperature of at least 0° C. According to another embodiment, freezing of the mitochondria is gradual. According to some embodiment, freezing of mitochondria is through flash-freezing. As used herein, the term “flash-freezing” refers to rapidly freezing the mitochondria by subjecting them to cryogenic temperatures.

In another embodiment, the mitochondria that underwent a freeze-thaw cycle were frozen for at least 30 minutes prior to thawing. According to another embodiment, the freeze-thaw cycle includes freezing the partially purified functional mitochondria for at least 30, 60, 90, 120, 180, 210 minutes prior to thawing. Each possibility represents a separate embodiment of the present invention. In another embodiment, the mitochondria that have undergone a freeze-thaw cycle were frozen for at least 1, 2, 3, 4, 5, 6, 7, 8, 9, 10, 24, 48, 72, 96, 120 hours prior to thawing. Each freezing time presents a separate embodiment of the present invention. In another embodiment, the mitochondria that have undergone a freeze-thaw cycle were frozen for at least 4, 5, 6, 7, 30, 60, 120, 365 days prior to thawing. Each freezing time presents a separate embodiment of the present invention. According to another embodiment, the freeze-thaw cycle includes freezing the partially purified functional mitochondria for at least 1, 2, 3 weeks prior to thawing. Each possibility represents a separate embodiment of the present invention. According to another embodiment, the freeze-thaw cycle includes freezing the partially purified functional mitochondria for at least 1, 2, 3, 4, 5, 6 months prior to thawing. Each possibility represents a separate embodiment of the present invention.

According to another embodiment, the mitochondria that underwent a freeze-thaw cycle were frozen within a freezing buffer. According to another embodiment, the mitochondria that underwent a freeze-thaw cycle were frozen within the isolation buffer. As used herein, the term “isolation buffer” refers to a buffer in which the mitochondria of the invention have been partially purified. In a non-limiting example, the isolation buffer includes 200 mM sucrose. 10 mM Tris-MOPS and 1 mM EGTA. According to some embodiments. BSA (Bovine Serum Albumin) is added to the isolation buffer during partial purification. According to some embodiments. 0.2% BSA is added to the isolation buffer during partial purification. According to some embodiments. HSA (Human Serum Albumin) is added to the isolation buffer during partial purification. According to some embodiments. 0.2% HSA is added to the isolation buffer during partial purification. According to other embodiment. HSA or BSA is washed away from the mitochondria of the invention following partial purification. Each possibility represents a separate embodiment of the present invention. Without wishing to be bound by any mechanism or theory, freezing mitochondria within the isolation buffer saves time and isolation steps, as there is no need to replace the isolation buffer with a freezing buffer prior to freezing or to replace the freezing buffer upon thawing.

According to another embodiment, the freezing buffer includes a cryoprotectant. According to some embodiments, the cryoprotectant is a saccharide, an oligosaccharide, or a polysaccharide. Each possibility represents a separate embodiment of the present invention. According to another embodiment, the saccharide concentration in the freezing buffer is a sufficient saccharide concentration which acts to preserve mitochondrial function. According to another embodiment, the isolation buffer includes a saccharide. According to another embodiment, the saccharide concentration in the isolation buffer is a sufficient saccharide concentration which acts to preserve mitochondrial function. According to another embodiment, the saccharide is sucrose. According to another embodiment, the saccharide is other than trehalose. Without wishing to be bound by any theory or mechanism, mitochondria that have been frozen within a freezing buffer or isolation buffer comprising sucrose demonstrate a comparable or higher oxygen consumption rate following thawing, as compared to control mitochondria that have not undergone a freeze-thaw cycle or that have been frozen within a freezing buffer or isolation buffer without sucrose.

According to some embodiments, addition of a saccharide to the mitochondria composition of the invention at a sufficient concentration acts to preserve mitochondrial function. According to another embodiment, a sufficient saccharide concentration which acts to preserve mitochondrial function is a concentration of between 100 mM-400 mM, preferably between 100 mM-250 mM, most preferably between 200 mM-250 mM. Each possibility represents a separate embodiment of the present invention. According to another embodiment, the saccharide according to the invention is sucrose. According to some embodiments the saccharide of the invention is other than trehalose. According to some embodiments the saccharide of the invention is other than mannitol.

According to another embodiment, the saccharide concentration in the composition of the invention is between 100 mM-150 mM. According to another embodiment, the saccharide concentration in the composition of the invention is between 150 mM-200 mM. According to another embodiment, the saccharide concentration in the composition of the invention is between 100 mM-200 mM. According to another embodiment, the saccharide concentration in the composition of the invention is between 100 mM-400 mM. According to another embodiment, the saccharide concentration in the composition of the invention is between 150 mM-400 mM. According to another embodiment, the saccharide concentration in the composition of the invention is between 200 mM-400 mM. According to another embodiment, the saccharide concentration in the composition of the invention is at least 100 mM. According to another embodiment, the saccharide concentration in the composition of the invention is at least 200 mM. Without wishing to be bound by any theory or mechanism of action, a saccharide concentration below 100 mM may not be sufficient to preserve mitochondrial function.

In some embodiments, the stimulant is heat aggregated-IgG.

Stimulated-platelets are centrifuged to remove remnant platelets or cells. The Supernatant containing the PEVs are then collected in step 160.

According to some embodiments, the PEVs are derived from the subject in need thereof. According to another embodiment, the PEVs are derived from a different subject than the subject in need thereof. According to another embodiment, the PEVs are derived from the same subject to whom they are administered. According to another embodiment, the PEVs are derived from a different subject than the subject to whom they are administered. According to another embodiment, the PEVs of the invention are from a source selected from autologous, allogeneic, and xenogeneic. Each possibility represents a separate embodiment of the present invention. As used herein, mitochondria of an autologous source refer to mitochondria derived from the same subject to be treated. As used herein, mitochondria of an allogeneic source refer to mitochondria derived from a different subject than the subject to be treated from the same species. As used herein, mitochondria of a xenogeneic source refer to mitochondria derived from a different subject than the subject to be treated from a different species. According to another embodiment, the PEVs of the invention are derived from a donor. According to some embodiments, the donor is an allogeneic donor. According to some embodiments, the donor is an autologous donor.

The term “subject in need thereof”, as used herein, refers to a subject afflicted with, or at a risk of being afflicted with, a condition which benefits from increased mitochondrial function. Each possibility represents a separate embodiment of the present invention. According to some embodiments. “a subject in need thereof” is a subject afflicted with a condition which may benefit from pro-apoptotic activity. In a non-limiting example, a condition which may benefit from pro-apoptotic activity is cancer. According to another embodiment, a subject in need thereof is mammalian. According to another embodiment, a subject in need thereof is human. According to another embodiment, a subject in need thereof is selected from the group consisting of: a human, a horse, a dog, a cat, a mouse, a rat, a cow and a sheep.

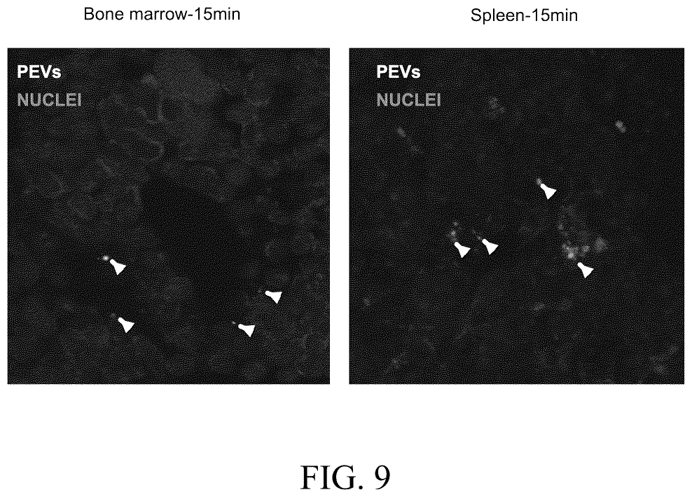

Some embodiments relate to a method of transducing platelet-derived mitochondria-containing extracellular vesicles (PEVs) into cells. The method includes 1) extracting PEVs from blood, and 2) incubating the PEVs with the cells for a time sufficient to transduce the PEVs into the cells. Incubating the cells can be in vitro or in vivo, the latter shown in FIG. 9.

In certain in vivo embodiments, incubating the PEVs with the cells includes injecting the PEVs into blood, cerebrospinal fluid, pleural fluid, pericardial fluid, peritoneal/ascitic fluid, synovial fluid, saliva, or any other bodily fluid of a subject. This can be accomplished in a variety of manners, including use of an appropriate catheter, such as an intra-arterial or intrathecal catheter. The PEVs can also be introduced into a specific organ or tissue of a subject, such as an eyeball or retina of the subject. For this purpose. PEVs can be delivered via intra-vitreal, intravenous, or intra-arterial injections.

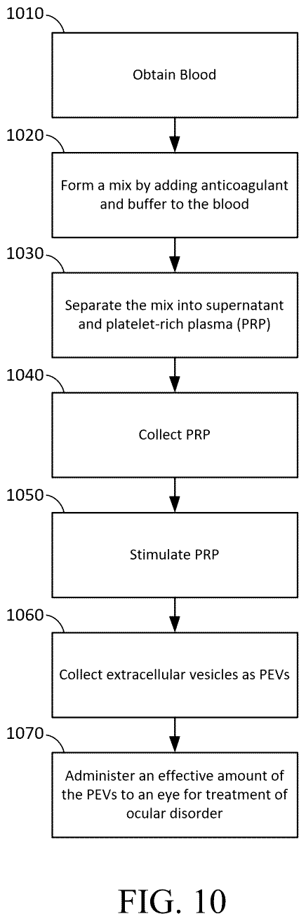

According to another embodiment, as shown in FIG. 10, a method of treatment for ocular disorders, or a symptom of the ocular disorder, in a patient in need thereof is provided, the method comprising obtaining platelet-derived extracellular vesicles that include mitochondria (PEVs). The PEVs have been collected by: obtaining blood from one or more donors in step 1010; adding an anticoagulant and a buffer to the blood to form a mix in step 1020; separating the mix into supernatant and platelet rich plasma (PRP) in step 1030; collecting the PRP in step 1040; stimulating the collected PRP in step 1050, thereby expelling extracellular vesicles from platelets in the PRP; and collecting the extracellular vesicles as PEVs in step 1060, many of these steps related to the collecting of the PEVs have been described in other embodiments, and thus, are similar. In some embodiments, the method further includes administering an effective amount of the PEVs to the eye of the patient in step 1070, thereby treating the ocular disorder. In some embodiments, the PEVs have been collected at a different site than a site where the treatment is carried out. In some embodiments, the PEVs are collected on-site or off-site.

As used herein, the term “on-site” refers to a location at which the administration step is performed or is to be performed. The location can be in the same room, office, or ward that the administration step is performed or is to be performed. The location can be in a same building that the administration step is performed or is to be performed. The location can be in the same building complex that includes a plurality of buildings, at least one of the plurality of buildings is where the administration step is performed or is to be performed. The building complex can have the same affiliation (business or organization) or at least one of the plurality of the buildings may have a different affiliation.

As used herein, the term “off-site” refers to an outside location that is apart from the location at which the administration step is performed or is to be performed. The outside location can be a room or a laboratory that is apart from the building and the building complex (if the building is part of the building complex) where the administration step is performed or is to be performed.

In some embodiments, the blood has been stored for about four days or more. In some embodiments, the blood has been stored for about one year. In some embodiments, the PEVs are frozen while stored. In some embodiments, the frozen PEVs are stored in combination with a cryoprotectant. In some embodiments, the cryoprotectant is selected from the group consisting of a saccharide, an oligosaccharide, and a polysaccharide. In some embodiments, the anticoagulant is anticoagulant citrate dextrose (ACD). In some embodiments, the buffer is Tyrode's buffer at about pH 6 to about pH 7, preferably at about pH 6.5. In some embodiments, the separating is conducted by centrifuge. In some embodiments, for the stimulating step, the collected PEVs are stimulated with immune complexes in the presence of Ca²⁺. In some embodiments, the immune complexes include heat aggregated-IgG. The concentration of the heat-aggregated Ig used in the stimulation step is preferably about 0.1 mg/mL to about 2.5 mg/mL, more preferably about 0.5 mg/mL. The concentration of the Ca²⁺ used in the stimulation step is about 1 mM to about 25 mM, more preferably about 5 mM. In some embodiments, the collected PRPs are stimulated by freeze-thaw cycles.

In some embodiments, after the administering the PEVs into the eye, the PEVs contact at least one cell of the eye. In some embodiments, the PEVs are internalized into the cell following the PEVs contacting the cell. As used herein, the terms “contact” and “contacting” refers to a composition, which includes mitochondria, that is in sufficient proximity to the cell to trigger internalization of at least the mitochondria into the cell.

In some embodiments, the effective amount for treatment of the ocular disorder corresponds to an amount of the internalized PEVs, which ranges from about 3 PEV/cell to about 100 PEV/cell for at least one cell as shown for RPECs and bENDs in FIGS. 6A-6B. In some embodiments, the effective amount corresponds to an amount of the internalized PEVs, which is about, for at least one cell. 3 PEV/cell, about 10 PEV/cell, about 30 PEV/cell or about 100 PEV/cell. The effective amount will vary, as recognized by those skilled in art, depending on the route of administration, possibility of co-administration with another therapeutic product(s), possibility of co-usage with another therapeutic treatment(s) or method(s), type(s) of delivery device(s) used, and usage of any excipients.

In some embodiments, the ocular disorder to be treated is aged macular degeneration (AMD). AMD is an ocular disorder that is one of the leading causes of vision loss, particularly in developed countries, having a prevalence of up to around 40%. AMD is characterized by mitochondrial dysfunction that affect the retina, this dysfunction brought upon by oxidative stress from reactive oxygen species (ROS). In AMD. ROS are produced at high levels in the RPE cells, which causes damage to mtDNA. The poor repair mechanisms of mtDNA allow this damage to accumulate over time to the point of causing the death of the mitochondria cells, which then leads to the death of the RPE cells. Because RPE cells support the health of photoreceptors, the death of the RPE cells lead to the demise of the photoreceptors that they support, which leads to visual loss.

In some embodiments, the ocular disorder to be treated is retinitis pigmentosa (RP). RP is an inherited disorder of the eye that causes severe vision impairment and is characterized by rod degeneration. In some embodiments, the ocular disorder is Leber's Hereditary Optical Neuropathy (LHON), a hereditary mitochondrial genetic disorder that is manifested by three primary mtDNA mutations in 90% of the cases. From these mtDNA mutations. LHON primarily affects retinal ganglion cells (RGC) causing their degeneration, which leads to vision loss. In some embodiments, the ocular disorder is diabetic retinopathy, which is characterized by the dysfunction of endothelial cells of the retinal microvasculature and the supporting cells of the retina such as Müller cells. In diabetic retinopathy, dysfunction of the endothelial cells leads to increased permeability thereof, which may bring about vascular leakage. This vascular leakage may cause edema in the surrounding, and thus, may lead to other relevant retinal diseases such as diabetic macular edema. In some embodiments, the ocular disorder is glaucoma.

In some embodiments, the cell to receive the PEVs includes a retinal pigment epithelium cell. In some embodiments, the cell to receive the PEVs includes a retinal ganglion cell. In some embodiments, the cell to receive the PEVs is located about a macula of the eye.

Specific tissues and organs can be specifically targeted by complexing the PEVs with specific receptors or coatings that facilitate “homing” to certain cell types. For example, U.S. Pat. No. 10,537,594, the contents of which are hereby incorporated by reference, exemplifies the use of asialoglycoprotein (AsG) receptor system to target mitochondria to liver cells. Similar systems can be used to target other tissues or organs.

Various techniques can be employed to facilitate internalization of mitochondria into cells both in vitro and in vivo. It is believed that high levels of mitochondrial internalization can be achieved for specific diseased tissues, or for the elderly, because in these cases the tissues are severely lacking in mitochondria. Cells that are energy-deficient will be expected to activate chemical pathways enabling easier internalization. When a free mitochondria or mitochondria-packed stem cell floats by, the cells signal their need and/or engulf the mitochondria. Even higher uptake of mitochondria can be obtained by adjusting timing, frequency, and duration so as to maintain large quantities of continuously in the bloodstream to ensure that there is always a ready supply. Precision placement can also be employed by injecting mitochondrial substance directly into an organ via an arterial shunt. For example, the hepatic artery can be used to concentrate mitochondria into the liver to preferentially regenerate liver tissue. By doing this properly, processes, such as clonal expansion, which threaten to dilute or reverse the mitochondria can be bypassed.

Diathermy and exercise by the subject can also facilitate uptake of mitochondria by cells. Exercise causes skeletal muscles to create more mitochondria. This is expected cause cells to accept more transplants. Research indicates this effect might be triggered also by heating the muscle with RF radio energy or ultrasound, for 2-4 hours per session.

Caloric restrictions/fasting by the subject can also facilitate uptake of mitochondria. Studies show that fasting causes mitochondrial changes, fission/fusion, or mitophagy. Perhaps some sequencing of fasts with transfusions would trick the cells into accepting a few extra mitochondria and more quickly share the new mtDNA with the mitochondrial network.

Other techniques can also be employed to facilitate to uptake of mitochondria. For example, partial poisoning, chemotherapy, or hypoxia to induce autophagy, followed by transfusion, repeated many times can be employed. In addition, metformin, melatonin, or other drugs are believed to stimulate uptake of mitochondria by cells. The use of drugs to shut down mitochondria's ability to reproduce itself by cloning can force the cells to rely only on transfused mitochondria.

Some embodiments relate to a method of increasing respiration of cells. The method includes transducing the cells with isolated PEVs according to the method described herein and producing ATP from the PEVs.

The isolated PEVs include functional mitochondria. In some embodiments, the term “functional mitochondria” refers to mitochondria that consume oxygen. In another embodiment, functional mitochondria have an intact outer membrane. Other embodiments include mitochondrial fragments, mitochondrial DNA, or segments thereof and mRNAs encoding mitochondrial gene products. In some embodiments, functional mitochondria are intact mitochondria. In another embodiment, functional mitochondria consume oxygen at an increasing rate over time. In another embodiment, the functionality of mitochondria is measured by oxygen consumption. In another embodiment, oxygen consumption of mitochondria may be measured by any method known in the art. According to some embodiments, functional mitochondria are mitochondria which display an increase in the rate of oxygen consumption in the presence of ADP and a substrate such as, but not limited to, glutamate, malate, or succinate. Each possibility represents a separate embodiment of the present invention. In another embodiment, functional mitochondria are mitochondria which produce ATP. In another embodiment, functional mitochondria are mitochondria capable of manufacturing their own RNAs and proteins and are self-reproducing structures. In another embodiment, functional mitochondria produce a mitochondrial ribosome and mitochondrial tRNA molecules.

As used herein, the term “intact mitochondria” refers to mitochondria comprising an outer and an inner membrane, an inter-membrane space, the cristae (formed by the inner membrane) and the matrix. In another embodiment, intact mitochondria include mitochondrial DNA. In another embodiment, intact mitochondria contain active respiratory chain complexes I-V embedded in the inner membrane. In another embodiment, intact mitochondria consume oxygen.

According to another embodiment, intactness of a mitochondrial membrane may be determined by any method known in the art. In a non-limiting example, intactness of a mitochondrial membrane is measured using the tetramethylrhodamine methyl ester (TMRM) or the tetramethylrhodamine ethyl ester (TMRE) fluorescent probes. Each possibility represents a separate embodiment of the present invention. Mitochondria that were observed under a microscope and show TMRM or TMRE staining have an intact mitochondrial outer membrane.

As used herein, the term “a mitochondrial membrane” refers to a mitochondrial membrane selected from the group consisting of: the mitochondrial inner membrane, the mitochondrial outer membrane or a combination thereof.

In some embodiments, the functional mitochondria are partially purified mitochondria. As used herein, the term “partially purified mitochondria” refers to mitochondria separated from other cellular components, wherein the weight of the mitochondria constitutes between 20-80%, preferably 30-80%, most preferably 40-70% of the combined weight of the mitochondria and other sub-cellular fractions (as exemplified in: Hartwig et al., Proteomics, 2009, (9):3209-3214), the disclosure of which is hereby incorporated by reference. Each possibility represents a separate embodiment of the present invention.

According to another embodiment, partially purified mitochondria do not contain intact cells. According to another embodiment, the composition of the invention does not include intact cells. According to another embodiment, the composition of the invention does not include mitochondrial clumps or aggregates or cellular debris or components larger than 5 μm. Each possibility represents a separate embodiment of the present invention. According to another embodiment, the composition of the invention is devoid of particulate matter greater than 5 μm. As used herein, the term “particulate matter” refers to intact cells, cell debris, aggregates of mitochondria, aggregates of cellular debris or a combination thereof. Each possibility represents a separate embodiment of the present invention. As used herein, a composition devoid of exogenous particulate matter greater than 5 μm includes no more than 1 μM of particulate matter greater than 5 μm, preferably less than 0.5 μM, most preferably less than 0.1 μM.

According to some embodiments, intact cells, cell debris or aggregates are removed from the composition of the invention. According to some embodiments, the composition of the invention is filtered through a filter of no more than 5 μm, in order to remove any intact cells, cell debris or aggregates, as exemplified herein below. Without wishing to be bound by any theory or mechanism, use of compositions comprising mitochondrial clumps according to the methods of the invention may be less efficient and even detrimental to the subject. According to another embodiment, the composition of the invention does not include liposomes or any other particulate carrier. Each possibility represents a separate embodiment of the present invention.

According to another embodiment, the weight of the mitochondria in partially purified mitochondria constitutes at least 20% of the combined weight of the mitochondria and other sub-cellular fractions. According to another embodiment, the weight of the mitochondria in partially purified mitochondria constitutes between 20%-40% of the combined weight of the mitochondria and other sub-cellular fractions. According to another embodiment, the weight of the mitochondria in partially purified mitochondria constitutes between 40%-80% of the combined weight of the mitochondria and other sub-cellular fractions. According to another embodiment, the weight of the mitochondria in partially purified mitochondria constitutes between 30%-70% of the combined weight of the mitochondria and other sub-cellular fractions. According to another embodiment, the weight of the mitochondria in partially purified mitochondria constitutes between 50%-70% of the combined weight of the mitochondria and other sub-cellular fractions. According to another embodiment, the weight of the mitochondria in partially purified mitochondria constitutes between 60%-70% of the combined weight of the mitochondria and other sub-cellular fractions. According to another embodiment, the weight of the mitochondria in partially purified mitochondria constitutes less than 80% of the combined weight of the mitochondria and other sub-cellular fractions.

The following examples are presented to provide a more complete understanding of the invention. The specific techniques, conditions, materials, proportions and reported data set forth to illustrate the principles of the invention are exemplary and should not be construed as limiting the scope of the invention.

### Example 1

This example illustrates that PEVs are collected from mouse blood according to some embodiments.

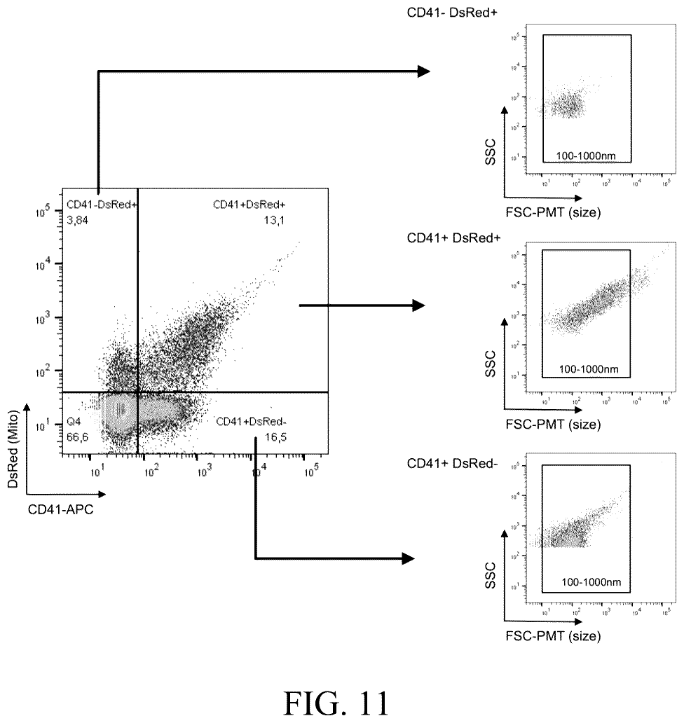

The following steps were conducted:
1. Blood was collected by from mouse donors. In this specific protocol, the donors were male DsRed mice, which are transgenic mice that express the red fluorescent protein variant DsRed. MST under the control of the chicken beta actin promoter coupled with the cytomegalovirus (CMV) immediate early enhancer. Here, 3×1 mL (1 mL/mouse) of blood was used.
2. ACD (20%) was added as an anticoagulant and 40% Tyrode's buffer pH 6.5 was also added to the blood. The blood mixture (20% ACD+40% Tyrode's buffer (TB) pH 6.5) was then centrifuged for 3 min at 500 g. PRP and buffy coat then collected and centrifuged for 2 min at 300 g.
3. PRP was collected and 20% ACD+10 mM EDTA added before a centrifugation step of 5 min at 1 300 g.
4. Each pellet was suspended in 0.1 mL TB pH 6.5 and 0.9 mL of TB pH 7.4 was added.
5. Platelets were pooled and counted using a cellometer and diluted at 10e8/mL in TB 7.4.
6. 900 millions of platelets were obtained in total and 5 mM CaCl2 was added prior stimulation.
7. Platelets were stimulated overnight (16 h) at room temperature with heat aggregated-IgG at 0.5 mg/mL. Heat aggregated-IgG was prepared by aggregating human IgG (25 mg/mL, MPBIO) at 62° C., for 1 hour.
8. 10 mM EDTA was added to stop the stimulation.
9. Stimulated-platelets were centrifuged at 300 g for 5 min to remove remnant platelets or cells.
10. Supernatant was collected and PEVs were analyzed using a flow cytometer.
11. Remaining platelets were evaluated and represented less than 1% contamination.
12. Obtained PEVs were diluted 3 times with PBS and centrifuged at 18 000 g for 90 minutes at 18° C.
13. Pellet was resuspended in 0.3 mL PBS and PEVs counted by flow cytometry. Concentration was estimated at 1.5×10e9 PEVs/ml.
14. PEVs may be tagged with CD41 tags to enable them to be counted in a flow cytometer. If so tagged, PEVs represented approximatively 40% of the total CD41+PEVs. Dotplot representing PEV populations are illustrated in FIG. 11. (DsRed axis indicates PEVs).

### Example 2

This example illustrates that PEVs can be internalized by retinal cells according to some embodiments.

The following steps were conducted:
1. Immortalized mouse retinal pigmented epithelial cells (RPEC) and brain endothelial cells (bEND) were plated overnight prior to PEV incubation, approximately 20,000 cells/well (RPEC) and 16,000 cells/well (bEND).
2. PEVs were collected from mouse donors following the steps outlined in Example 1.
3. RPECs were preincubated with or without PEVs (about 3, 10, 30, or 100 mitochondria+PEVs per cell) for either 3, 18, 24, or 36 hours in Prigrow III, supplemented with 1% Pen-Strep (pH 7.4) and 1%, 5%, or 10% FBS (non-heat activated).
4, bENDs were preincubated with or without PEVs (about 3, 10, 30, or 100 mitochondria+PEVs per cell) for 24 hours in DMEM, supplemented with 1% Pen-Strep (pH 7.4) and 1%, 5%, or 10% FBS (non-heat activated).
5. RPECs and bENDs were washed and put in XF medium supplemented with 2 mM glutamine. 1 mM pyruvate, and 8 mM D-glucose and 1% FBS at a pH of 7.4.
6. After the wash, the RPECs and bENDs in XF medium were centrifuged at 300 g for 5 min and 60 mins at 37° C., without CO₂.

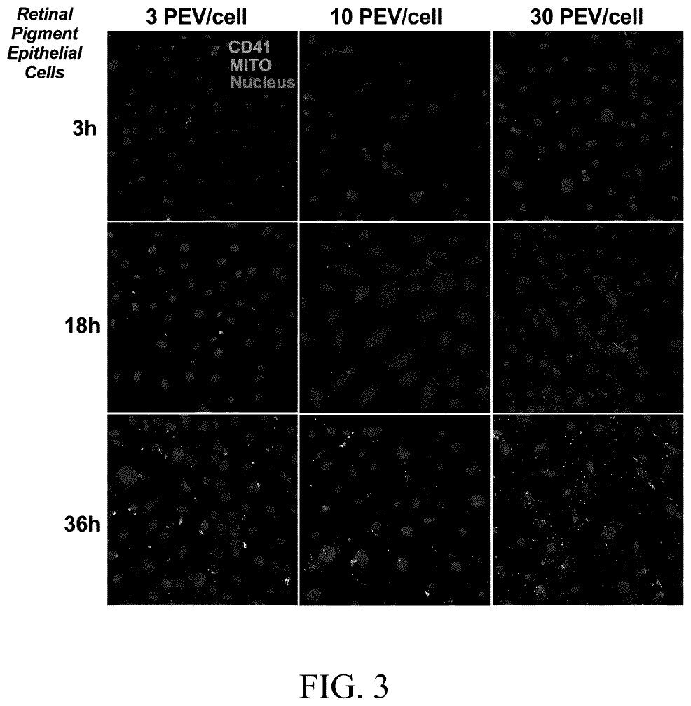

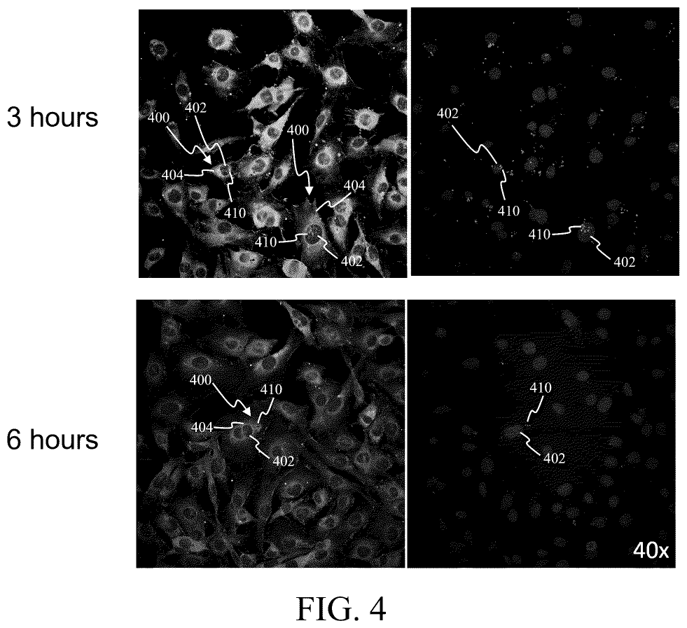

FIGS. 2A-2B are confocal images that show stained nuclei 202 [DAPI (4′,6-diamidino-2-phenylindole)] and cellular membranes 204 of RPECs 200, and PEVs 210 (DsRed). As shown in FIGS. 2A-2B (40× and 20× magnification respectively), the PEVs 210 are largely internalized by the RPECs 200. As shown in FIG. 3, this internalization may be stable for well over 24 hours. FIG. 4 shows another group of RPECs 400, their nuclei 402 stained with DAPI, demonstrating their ability to uptake administered PEVs/mitlets 410 (DsRed) after three and six hours. The left panels of FIG. 4 show the RPECs 400, including their stained nuclei 402 and their cell membranes 404. Also shown in the left panels are the stained PEVs/mitlets 410. The right panels of FIG. 4 show: 1) the stained nuclei 402 of the RPECs 400 from adjacent the left panel; and 2) the stained PEVs/mitlets 410 from the adjacent left panel. The panels from FIG. 4 were taken at 40×.

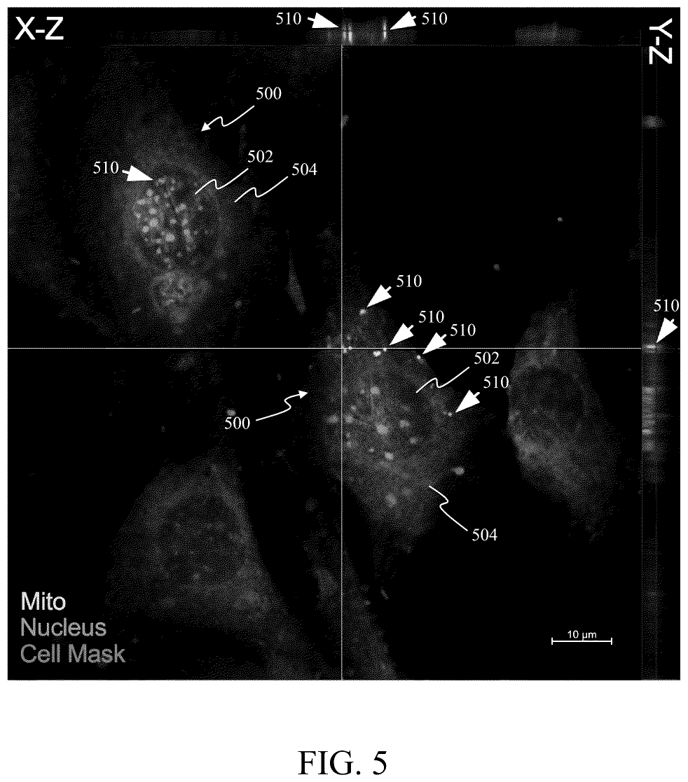

To verify mitochondria internalization in RPECs 500. X-Z and Y-Z scans of fluorescent-labeled mitochondria 510 (indicated by the labeled arrowheads here) from PEVs were performed using a confocal microscope as shown in FIG. 5. The nucleus 502 of the RPECs 500 were stained with DAPI. The cell membrane 504 of the RPECs 500 were stained with CellMask™ as indicated in FIG. 5. Here, the X-Z plane is perpendicular to the Y-Z plane. The X-Z. Y-Z scans show how the highest intensity from the point source of the fluorescently labeled mitochondria 510 (see arrowheads) is located within the RPEC 500. The results of this internalization are shown in FIG. 6A. FIG. 6B shows that bENDs also internalized mitochondria that were delivered by PEVs. Accordingly, these results demonstrate that PEVs can deliver durable mitochondria into RPECs and bENDs.

To assess the activity of the mitochondria delivered by PEVs, a Seahorse XF Assay that tests for mitochondria stress was performed on the RPECs that were preincubated with PEVs for 24 hours. The oxygen consumption rate (OCR) was measured between different preincubation parameters for RPEC (0, 3, 10, 30, or 100 PEVs/cell) as provided in step 3 of this example with 5% FBS appearing to be the preferred level of serum used: higher serum levels like 10% FBS may trigger cellular division.

With respect to the OCR readings of FIGS. 7A-7D, similar (if not slightly lower) basal respiration levels between the RPECs that were preincubated with PEVs and those that were not (controls), are shown in FIG. 7A. Basal respiration levels refer to the energetic demand of the RPECs under baseline conditions. Increased spare respiratory capacity of RPECs that were preincubated with PEVs is shown in FIG. 7B, the results of which indicate improvement in the capability of the RPEC to respond to an energic demand (i.e. improved cell fitness or flexibility). FIG. 7C appears to show slightly enhanced ATP production in some of the RPECs that were preincubated with PEVs. FIG. 7D shows that proton leak does not appear to be an issue with RPECs that internalized PEVs compared to those that did not.

### Example 3

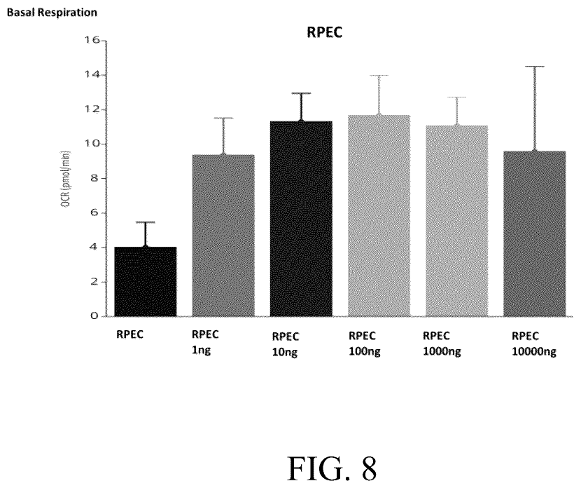

Naked mitochondria were extracted from raw liver and administered to RPECs at various amounts in separate wells: 1, 10, 100, 1000, and 10000 ng. The oxygen consumption rate for each group was determined by the Seahorse XF assay. As shown in FIG. 8, each group of RPECs that were administered naked mitochondria displayed significant increases to their respective basal respiration compared to the control RPECs that did not receive any naked mitochondria, the control RPECs showing an OCR of about 4 pmol/min. As shown, the increase in the basal respiration of RPECs that received naked mitochondria was at least two-fold compared to the controls.

### Additional Notes

The section headings used herein are for organizational purposes only and are not to be construed as limiting the subject matter described.

As used herein, term “and/or” shall be taken to provide explicit support for both meanings or for either meaning.

As used herein, the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The following explanations of terms and methods are provided to better describe the present disclosure and to guide those of ordinary skill in the art in the practice of the present disclosure. The singular forms “a.” “an,” and “the” refer to one or more than one, unless the context clearly dictates otherwise. For example, the term “comprising a nucleic acid molecule” includes single or plural nucleic acid molecules and is considered equivalent to the phrase “comprising at least one nucleic acid molecule.” The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements. Unless otherwise specified, the definitions provided herein control when the present definitions may be different from other possible definitions.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

Reference throughout the specification to “one example”, “another example”, “an example”, and so forth, means that a particular element (e.g., feature, structure, and/or characteristic) described in connection with the example is included in at least one example described herein, and may or may not be present in other examples. In addition, it is to be understood that the described elements for any example may be combined in any suitable manner in the various examples unless the context clearly dictates otherwise. While several examples have been described in detail, it is to be understood that the disclosed examples may be modified. Therefore, the foregoing description is to be considered non-limiting.

Features, materials, characteristics, or groups described in conjunction with a particular aspect, or example are to be understood to be applicable to any other aspect or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing examples. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable sub-combination. Moreover, although features may be described above as acting in certain combinations, one or more features from a claimed combination can, in some cases, be excised from the combination, and the combination may be claimed as a sub-combination or variation of a sub-combination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some examples, the actual steps taken in the processes illustrated and/or disclosed may differ from those shown in the figures. Depending on the example, certain of the steps described above may be removed or others may be added. Furthermore, the features and attributes of the specific examples disclosed above may be combined in different ways to form additional examples, all of which fall within the scope of the present disclosure.

For purposes of this disclosure, certain aspects, advantages, and novel features are described herein. Not necessarily all such advantages may be achieved in accordance with any particular example. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain examples include, while other examples do not include, certain features, elements, and/or steps. Thus, such conditional language is not generally intended to imply that features, elements, and/or steps are in any way required for one or more examples or that one or more examples necessarily include logic for deciding, with or without user input or prompting, whether these features, elements, and/or steps are included or are to be performed in any particular example.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain examples require the presence of at least one of X, at least one of Y, and at least one of Z.

Language of degree used herein, such as the terms “approximately,” “about,” “generally,” and “substantially” represent a value, amount, or characteristic close to the stated value, amount, or characteristic that still performs a desired function or achieves a desired result.

The scope of the present disclosure is not intended to be limited by the specific disclosures of preferred examples in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

The described embodiments and examples of the present disclosure are intended to be illustrative rather than restrictive, and are not intended to represent every embodiment or example of the present disclosure, and thus, are not to be limited in scope by the specific embodiments and examples described herein. While the fundamental novel features of the disclosure as applied to various specific embodiments thereof have been shown, described, and pointed out, it will also be understood that various omissions, substitutions, and changes in the details of the methods that are disclosed, may become apparent and may be made by those skilled in the art without departing from the spirit of the disclosure. For example, it is expressly intended that all combinations of those method steps that perform substantially the same function in substantially the same way to achieve the same results are within the scope of the disclosure. Moreover, it should be recognized that method steps shown and/or described in connection with any disclosed form or embodiment of the disclosure may be incorporated in any other disclosed or described or suggested form or embodiment as a general matter of design choice. Further, various modifications and variations can be made without departing from the spirit or scope of the disclosure as set forth in the following claims both literally and in equivalents recognized in law.

What is claimed is:

1. A composition comprising platelet-derived extracellular vesicle that include mitochondria (PEV) for treating an ocular disorder, or a symptom of the ocular disorder, wherein the PEVs have been collected by:
obtaining blood from one or more donors;
adding an anticoagulant and a buffer to the blood to form a mix;
separating the mix into supernatant and platelet rich plasma (PRP);
collecting the PRP;
stimulating the collected PRP, thereby expelling extracellular vesicles from platelets in the PRP; and
collecting the extracellular vesicles as the PEVs.

2. The composition according to claim 1, wherein the PEVs have been collected at a different site than a site where the treatment is carried out.

3. The composition according to claim 1, wherein the PEVs are provided in a syringe that is configured to deliver the PEVs into the eye.

4. The composition according to claim 1, wherein the PEVs are provided in a port delivery system that provides a PEV implant for prolonged delivery of the PEVs into the eye.

5. The composition according to claim 1, wherein the PEVs are administered into a vitreous chamber of the eye.

6. The composition according to claim 1, wherein the collected PRP is stimulated with immune complexes in presence of Ca²⁺.

7. The composition according to claim 6, wherein the immune complexes comprise heat-aggregated IgG.

8. The composition according to claim 1, wherein the collected PRP is stimulated by freeze-thaw cycles.

9. The composition according to claim 7, wherein concentration of the heat-aggregated IgG is about 0.1 mg/mL to about 2.5 mg/mL, and wherein concentration of the Ca²⁺ is about 1 mM to about 25 mM.

10. The composition according to claim 1, wherein the anticoagulant is anticoagulant citrate dextrose (ACD).

11. The composition according to claim 1, wherein the buffer is Tyrode's buffer at about pH 6 to about pH 7.

12. The composition according to claim 1, wherein the separating step is conducted by centrifuge.

13. The composition according to claim 1, wherein the blood has been stored for four or more days.

14. The composition according to claim 13, wherein the blood has been stored for up to one year.

15. The composition according to claim 1, wherein the PEVs are administered in a manner that the PEVs contact at least one cell of the eye.

16. The composition according to claim 15, wherein the PEVs are internalized into the cell after the PEVs contact the cell.

17. The composition according to claim 16, wherein the PEVs are administered at a dosage of about 3 PEV/cell to about 100 PEV/cell.

18. The composition according to claim 13, wherein the PEVs are frozen while stored.

19. The composition according to claim 18, wherein the frozen PEVs are stored in combination with a cryoprotectant.

20. The composition according to claim 19, wherein the cryoprotectant is selected from the group consisting of a saccharide, an oligosaccharide, and a polysaccharide.
